(19) United States
(12) Patent Application Publication
(10) Pub. No.: US 2011/0029009 A1
(43) Pub. Date: Feb. 3, 2011

(54) Authorized Firearm Bearer Identification System

(76) Inventor: Richard Dietl, New York, NY (US)

Correspondence Address: MICHAELSON & ASSOCIATES, P.O. BOX 8489, RED BANK, NJ, 07701-8489, US

(21) Appl. No.: 12/842,081

(22) Filed: Jul. 23, 2010

Related U.S. Application Data
(60) Provisional application No. 61/229,066, filed on Jul. 28, 2009.

(51) Int. Cl. A61B 17/00; F41C 33/02

(52) U.S. Cl. 606/203; 40/299.01; 40/662; 40/672; 40/1.5; 224/243

(57) ABSTRACT

An identification system and method that enables third parties, such as uniformed police officers, to identify another authorized firearms bearer, in order reduce risk of misidentification of intent that might otherwise lead to an unfortunate accidental injury during an incident response. The system includes an identification tag that is selectively affixable to a firearm in a position visible to third parties. The identification tag is selectively affixed in a stored position that preferably is furled so that it does not interfere with firearms storage or usage. The tag is deployable in an extended position to increase potential recognition to third parties. The tag desirably may also be wearable by the firearms bearer, such as a wrist band, head band or sash.

### CLAIM TO PRIORITY

This application claims the benefit of co-pending U.S. provisional patent application entitled “Authorized Firearm Bearer Identification System filed Jul. 28, 2009 and assigned Ser. No. 61/229,066, which is incorporated by reference herein.

### BACKGROUND OF THE DISCLOSURE

1. Field of the Invention

The invention relates to systems and methods to identify authorized firearm bearers, such as police officers, retired police officers, security guards and investigation personnel, concealed firearms carry permit holders and military personnel. The systems and methods are intended to help reduce risk of misidentification and accidental shooting of firearms bearers during response to a potentially violent incident, often referred to as fratricide.

2. Description of the Prior Art

During responses to potentially violent incidents by police or military personnel, respondents may have to make split-second decisions whether another firearms bearer is “friendly” or a “hostile” threat. An identification error may lead to an unfortunate accidental shooting of an innocent person who had no intent to harm the shooter and may in fact be friendly and supportive of the shooter's goals and objectives. News reports are replete with incidents of uniformed police officers and soldiers accidentally shooting fellow comrade personnel due to misidentification during the early split-seconds of their mutual encounter. While no identification system can replace the professional judgment of a responding police officer of soldier who is on the scene, such an identification system may give respondents additional on-the scene information that can influence the respondents' shoot or no-shoot decision. For brevity, further discussion of past identification systems will be in the context of laws enforcement personnel.

Police officers have worn duty uniforms recognizable by the public and other law enforcement officers. A responding officer is trained not to shoot a fellow uniformed officer during a potentially violent response call, even when both officers have drawn their firearms. Identification becomes more complex when some of the responding officers are on plain-clothes or undercover assignments, or are off duty out of uniform but otherwise legally required to respond to criminal incidents. Identification complexities are further compounded if a non-uniformed armed individual displaying a firearm is a retired police officer, an officer from a different law enforcement agency or a legally armed private citizen with a concealed firearms carry permit who is legitimately acting in self defense.

In the past, police agencies have attempted to equip officers who are not wearing duty uniforms with selectively wearable identification clothing having concealable visual identification panels in hidden pockets, removable hats, badges, hook and loop affixable patches (e.g., bearing the legend POLICE, FEDERAL AGENT or the like). Undercover police officers who could not risk having in their possession for fear of discovery by criminals any garments that would otherwise identify them as police officers would sometimes wear color coded garments or accessories, or wear objects at specific locations on their persons that would be a pre-arranged identification code to their fellow police officers.

Any specific-purpose garment or accessory runs the risk of being left behind by a police officer at any moment of necessary response. A pre-arranged identification code that is garment specific is useless if the responding officer does not have that particular garment or is not aware of the color code/secret garment wearing location on the given response day. If the non-uniformed officer displays a firearm during an incident response, there is greater risk that he or she may be misidentified by other responding officers as a violent criminal rather than as a fellow police officer.

In the past some police agencies have marked their firearms with visible coded indicia recognizable by other responding police officers, such as colored paint or tape stripes on barrels. In low light conditions often encountered in police work (night criminal activities or in building interiors) such visual indicia may not be sufficiently large to be visible by other responding officers. Also indicia stripes on barrels may not be visible on a handgun retained in a holster of a plain clothes or undercover officer. In such situations only the handgun butt is visible.

Thus, a need exists in the art for an authorized firearm bearer identification system that is intended to be stored directly on the firearm and deployable when the firearm is displayed, so that other responding officers instantly visually associate the firearm with another “friendly” fellow police officer. In this way the primary act that causes concern to police officers, namely display of a firearm, can mitigate fellow officer concerns when the identification tag is deployed directly on the firearm or alternatively worn by the officer. In this manner, initial officer anxiety about the presence of a stranger with a firearm that causes the officer to focus visual and mental attention on the stranger's firearm is mitigated by a “friendly” equally visual identification signal.

### SUMMARY OF THE INVENTION

Accordingly, an object of the invention is to create a firearm bearer identification system that is stored directly on the firearm, so that it is present when the firearm user displays the firearm. The identification system is stored in a configuration that minimizes interference with firearm manipulation, for example avoiding the tendency to snag on surrounding objects. When the firearm is displayed the identification tag of the present invention can be deployed in a more visible configuration on the firearm, or alternatively worn by the firearm bearer as, for example, a head band, arm band, waist or chest sash, or affixed to other clothing.

These and other objects are achieved in accordance with the present invention by a firearm bearer identification system including a firearm; and an identification tag that is selectively affixable to the firearm in a stored position wrapped about the firearm or in a storage compartment coupled to the firearm. The tag is extendable from the firearm in a deployed position. Preferably the tag is visible while the firearm is stored, but does not interfere with firearm storage. For example the tag may be stored in a furled configuration on the grip portion of a holstered handgun or the grip portion of a long gun that is stowed in a locked unmarked patrol car rack. The furled identification tag in either stored firearm situation would be visible to a uniformed police officer. When the authorized user needs to display the firearm in public the identification tag may be unfurled to deploy it in a more visible configuration. If preferred by the officer or departmental policy guidelines, in an alternative embodiment of the present invention at least a portion of the identification tag may be removed and worn as a garment.

The identification tag of the present invention may be constructed of resilient, stretchable material, or with a surface that enhances grip by the firearm bearer in wet (e.g., environmental, perspiration or body fluids) or cold conditions. The identification tag of the present invention may form a loop bight for passage and retention of a portion of a firearm and may have a hook and loop fastening system construction to aid in forming a tight loop as well as for unfurling the tag in selected positions. The tag surface may be reflective of light or to enhance visual contrast for imaging enhancement systems, e.g., night vision or thermal imaging systems. The tag desirably may include identification indicia, such as for example POLICE, SECURITY or law enforcement agency name. The tag may include a stiffening member that is furlable in a stored position and extendable in a deployed position.

In case of emergency trauma, such as a gunshot wound or compound fracture with arterial bleeding, an embodiment of the identification tag of the present invention may be wrapped around the limb of an injured person as a tourniquet.

The objects and features of the present invention may be utilized jointly or severally in any desired combination.

### BRIEF DESCRIPTION OF THE DRAWINGS

The teachings of the present invention can be readily understood by considering the following detailed description in conjunction with the accompanying drawings, in which:

FIG. 1 shows the firearms identification system of the present invention, including an identification tag in a furled storage position about the grip portion of a revolver that is inserted in a holster, with the holster drawn in phantom;

FIG. 2 shows the firearms identification system of FIG. 1, with the identification tag unfurled in a deployed position;

FIG. 3 is a view similar to that of FIG. 3, wherein an authorized firearms user has deployed the identification tag from the grip portion of a semi automatic pistol;

FIG. 4 shows firearms identification tags of the present invention representatively affixed to the butt stock and barrel portions of a shotgun long gun;

FIG. 5 shows representative attachment locations of the identification tag of the present invention on the body of an authorized firearms user;

FIGS. 6 and 7 are partial planar views of both sides of the identification tag of the present invention;

FIG. 8 is a cross sectional view of the identification tag of the present invention taken along 8-8 of FIG. 6;

FIG. 9 shows a representative alternative construction of the identification tag of the present invention showing formation of a looped bight formed in the tag for receipt of a firearm therein; and

FIG. 10 shows the identification tag of the present invention being utilized as a tourniquet about an injured patient's limb.

To facilitate understanding, identical reference numerals have been used, where possible, to designate identical elements that are common to the figures.

### DETAILED DESCRIPTION

After considering the following description, those skilled in the art will clearly realize that the teachings of my invention can be readily utilized in an authorized firearm user identification system.

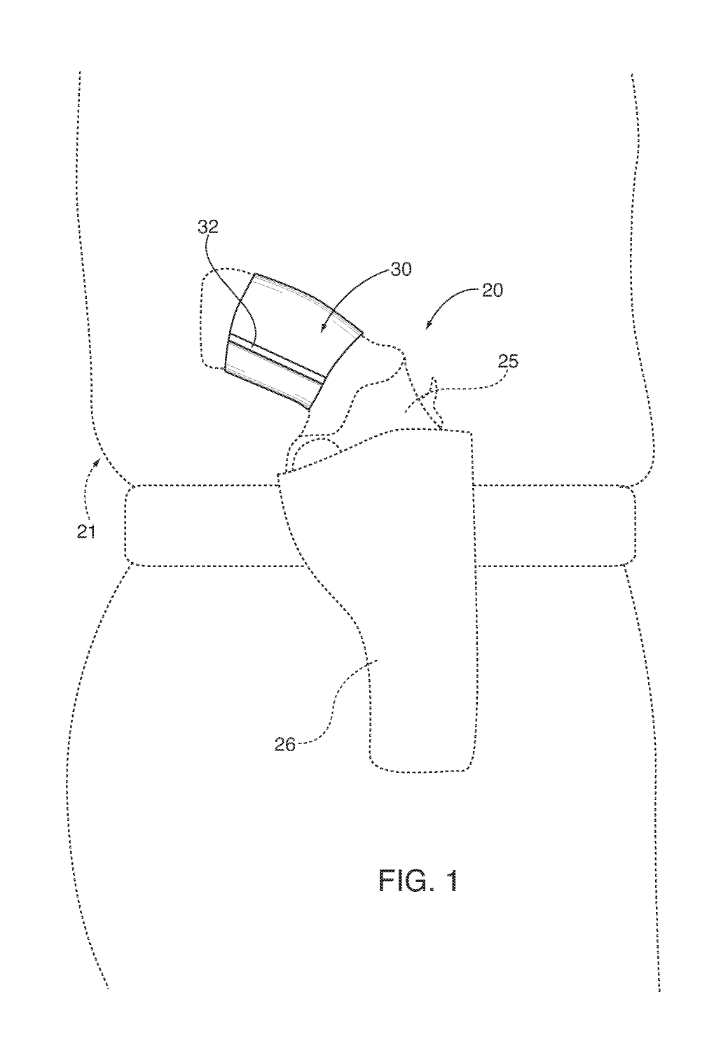

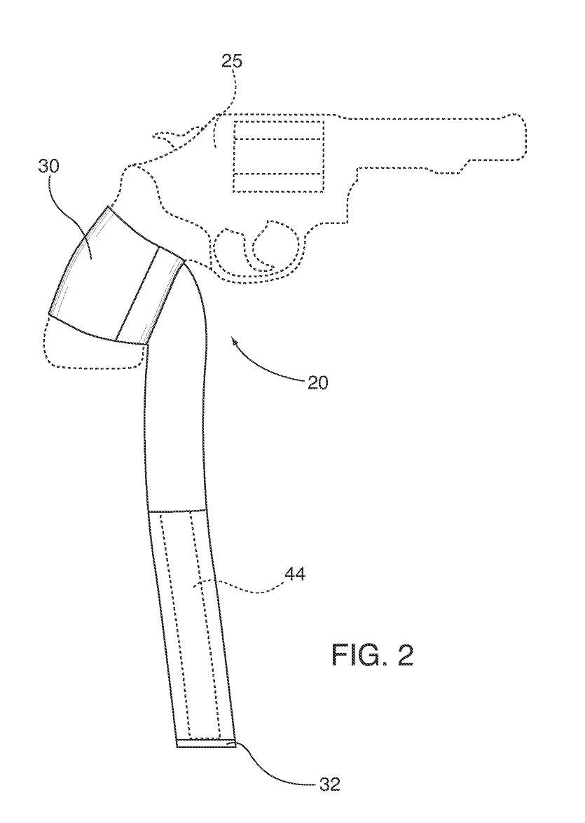

FIGS. 1 and 2 show the firearms identification system 20 (hereafter sometimes referred to as “FID system”) of the present invention, as worn by an authorized firearms user 21. The FID system 20 includes a firearm, such as revolver 25 shown retained in holster 26 (shown in schematic phantom view) and a firearms identification tag 30 (hereafter sometimes referred to as “FID tag”) which may advantageously be in the form of an elongated strip. The FID tag 30 has a distal end portion 32.

As shown in FIG. 1 the FID tag 30 is in a furled, stored position wrapped about the grip portion of the revolver 25 so that it does not get inadvertently snagged about other objects. It should be noted that the FID tag 30 alternatively may be furled by bunching, pleating or any other way of otherwise gathering excess length of the strip. When the revolver 25 is retained within the holster 26 the FID tag visibly protrudes from the grip portion. Should the authorized firearm user 21 visibly expose the revolver 25 intentionally or inadvertently while in the holster 26, other police officers would be able to see the FID tag 30 and recognize that the firearm is being worn by an authorized user. This would be a valuable piece of tactical information that the firearm wearer 21 is “friendly” and should, not be confused with a violent offender.

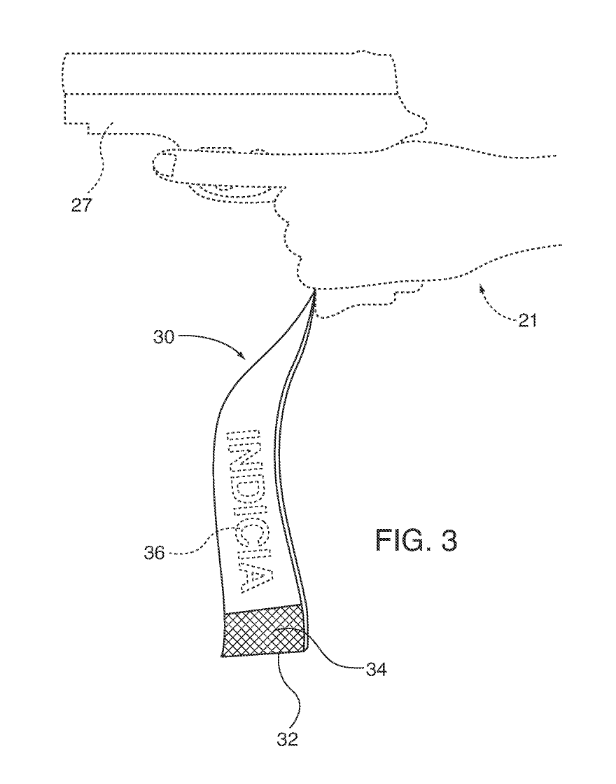

In FIG. 2 the FID tag 30 is shown in unfurled, deployed position with the tag distal end 32 extending from the revolver 25. The authorized user 21 could selectively unfurl/deploy the FID tag 30 while the revolver is in its holster 26 or alternatively while being held in the user's hand. FIG. 3 shows the FID tag 30 in its unfurled deployed position while the user 21 has obtained a firing grip with trigger finger outside the trigger guard of semi-automatic pistol 27. The FID tag 30 is shown with optional reflective portion 34 to aid in visual recognition by the human eye or by electronic imaging equipment (e.g., night vision goggles or thermal imaging scopes) under reduced lighting conditions. The reflective portion 34 may cover any portion of, or the entire FID tag strip, and may be integrally formed, bonded or otherwise affixed to the strip material. Other identification indicia 36, such as for example words (POLICE) or other symbols may be affixed to the FID tag 30 strips. The indicial 36 may also be formed of reflective material. The strip or indicia may be manufactured in any color or surface finish.

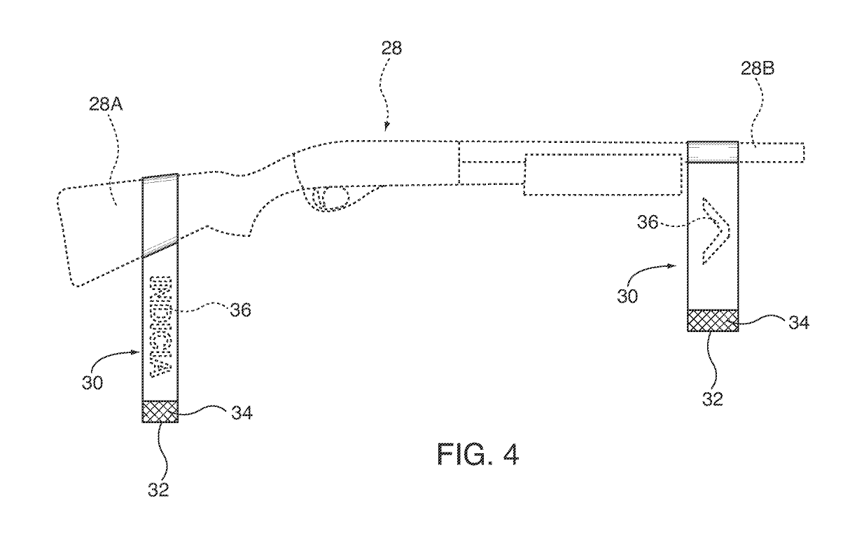

FIG. 4 shows exemplary utilization of the FID tags 30 in a deployed configuration on a shotgun long gun firearm. The tags 30 may be selectively affixed to any desired portion of the shotgun, such as on the butt stock 28A, the barrel/magazine tube 28B forward of the reciprocating pump fore end, as well as the butt stock pistol grip portion (no tag shown in that position). Note that identification indicia 36 on the barrel 28B FID tag 30 is in the form of the Greek letter lambda (A) occasionally utilized by military units to designate “friendly” forces. By analogy the previous examples of a handgun retained within in a holster with the FID tag oriented in a visible position, one can appreciate that the FID tag can be oriented on a long gun in a visible position while the rifle or shotgun is retained within a case or scabbard. For brevity, the term “holster” is intended to encompass all forms of firearms cases, scabbards or storage devices.

One skilled in the art can appreciate that the FID tag of the present invention can be employed with other types of handguns, long guns or firearms, including semi or fully automatic rifles, or military squad weapons. While the figures herein show the exemplary FID tag embodiments wrapped about firearms, one skilled in the art can appreciate that FID tags of the present invention can also be stored in compartments directly or indirectly affixed to the firearms.

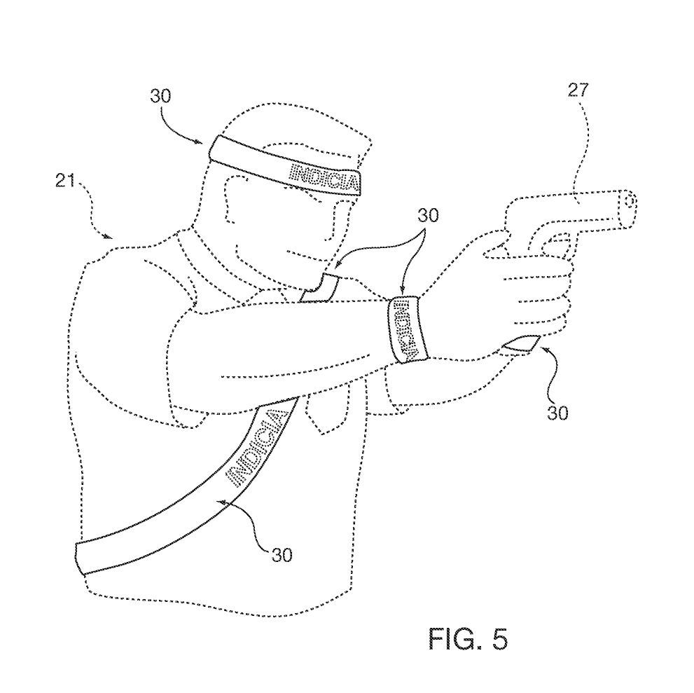

When the authorized firearm user 21 wishes to remove all or a portion of the FID tag 30, it may be utilized as a garment or other wearable tag, whether or not the firearm 27 is in its holster or held as shown in FIG. 5. Advantageously this enables an authorized user to wear the FID tag 30 selectively in a very visual manner when responding to a crime incident without added risk of being accidentally shot by another police officer if the firearm were to be displayed. As shown in FIG. 5 the authorized user police officer 21 can selectively wear the FID tag 30 as a head band, sash or armband. Also, if the FID tag 30 has a hook and loop fastening system, it may be affixed directly to any garment (e.g., shirt or jacket) that has a corresponding mating portion of the fastening system. Advantageously the FID tag 30 may be constructed of multiple attached portions so that as shown in FIG. 5 one portion of the tag may be worn as a garment while a portion of the tag remains attached to the semi automatic pistol firearm 27.

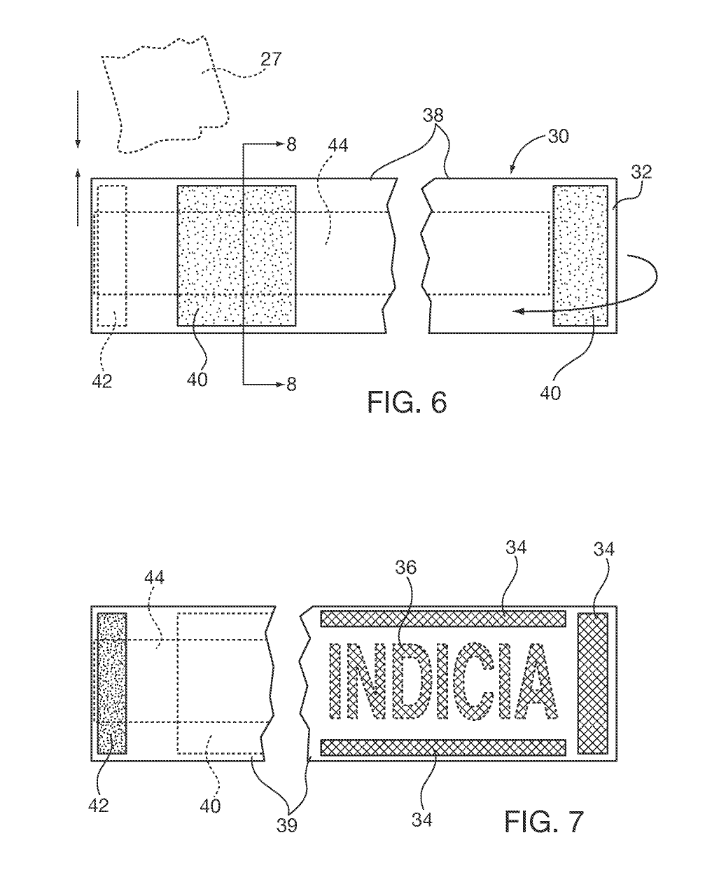

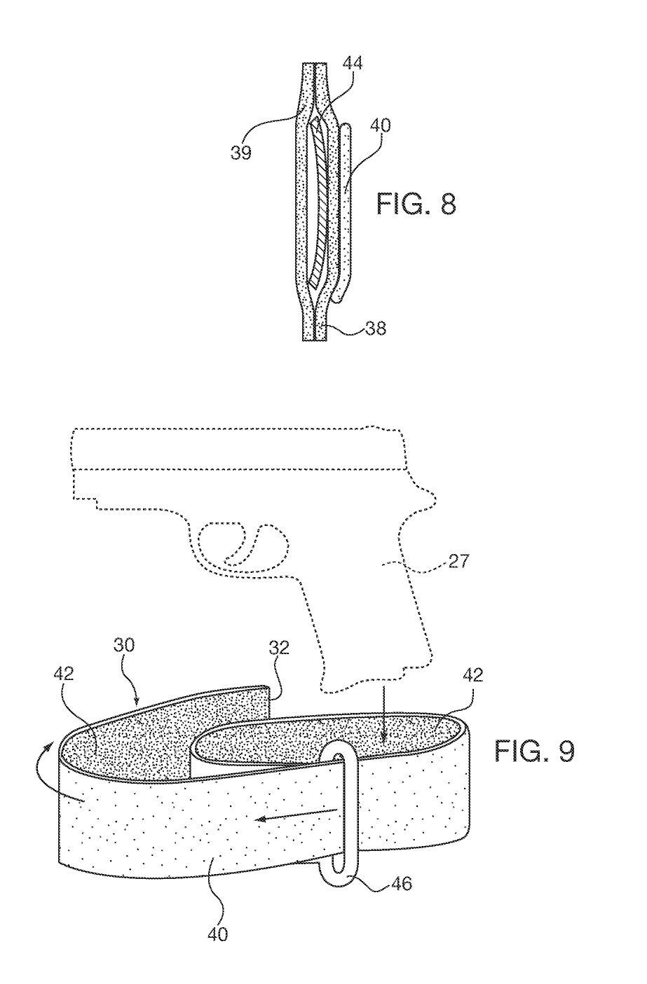

FIGS. 6-9 show exemplary construction features of the FID tag 30 of the present invention. It should be noted that the structural features may be selectively employed in whole or any sub combination. The FID tag 30 has strips of opposing material 38, 39 between which may be sandwiched a resilient stiffening member 44. The stiffening member allows the FID tag 30 to be furled in a wrapped and rolled stored configuration about the grip portion of exemplary semi automatic pistol 27, yet provides stiffness when in the unfurled display position. If desired the stiffening member 44 may be constructed or arched cross section spring steel of the type used in tape measures. Alternatively the stiffening member 44 may be embedded within the FID tag 30. The stiffening member 44 is not necessary to practice all embodiments of the present invention. If desired, it may be eliminated and a single strip of material 38 may be utilized to construct the FID tag 30.

The FID tag 30 may include opposed mating hook and loop fastening elements 40, 42 that may be affixed to the strips 38, 39 as shown in FIGS. 6-8. Alternatively, as shown in FIG. 9, the entire FID tag 30 may comprise a unitary strip of hook and loop fastener, with the hook elements on one side and the loop elements on the opposite side. FIG. 9 also shows an additional optional construction feature, wherein eye hook 46 is formed within the FID tag 30 strip. The distal end 32 of the tag strip is passed through the eye hook 46 in order to form a loop bight for insertion of a semi automatic pistol 27 grip portion. When the loop bight is cinched about the circumference of the pistol grip portion it provides tight compression and reduces the likelihood that the FID tag will slide off the pistol 27. The remainder of the hook and loop strip is wrapped about the grip portion, the mating contact of the strip upon itself reducing the likelihood that the strip will become inadvertently unwrapped.

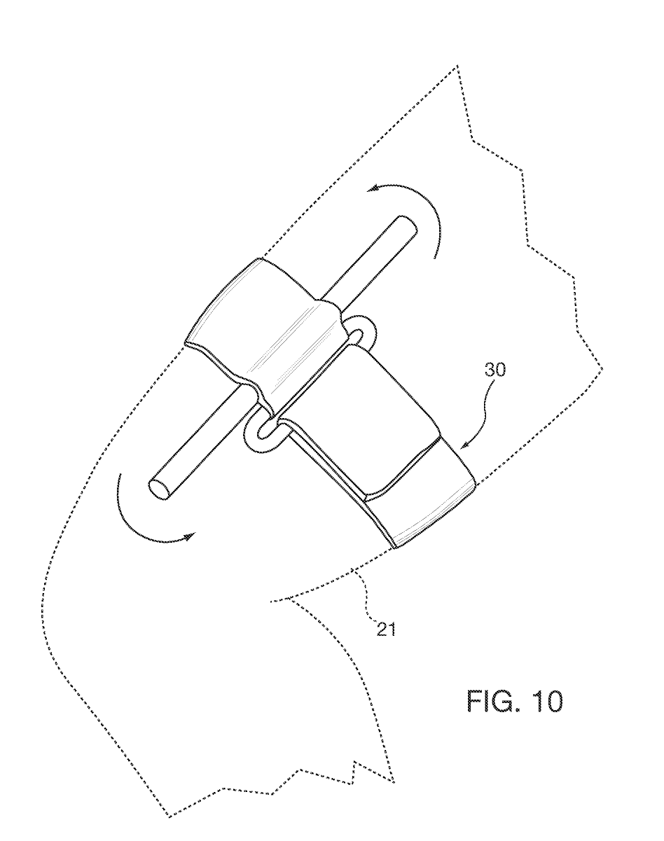

As shown in FIG. 10 the FID tag 30 of the present invention may be utilized as a medical tourniquet by cinching it about a user/patient's limb extremity, wrapping excess strip around itself and inserting a tourniquet tensioning rod 47. The rod 47 is twisted parallel to the surface of the patient's limb to compress the strip and reduce blood flow to the extremity distal the tourniquet application point.

Although various embodiments which incorporate the teachings of the present invention have been shown and described in detail herein, those skilled in the art can readily devise many other varied embodiments that still incorporate these teachings.

What is claimed is:

1. An authorized firearm bearer identification system, comprising:
a firearm; and
an identification tag that is selectively affixable to the firearm in a stored position and a deployed position visibly extending from the firearm.

2. The firearm identification system of claim 1, wherein the tag is furled by wrapping about a portion of the firearm in the stored position.

3. The firearm identification system of claim 1, wherein at least a portion of the tag is wearable by the firearms bearer.

4. The firearm identification system of claim 1, further comprising a firearm retention holster, and wherein the identification tag is affixed to the firearm in a visible position when the firearm is retained by the holster.

5. The firearm identification system of claim 1, wherein the identification tag comprises a furlable strip.

6. The firearm identification system of claim 5, wherein the strip forms a bight for passage and retention of the firearm therein.

7. The firearm identification system of claim 1 wherein the tag comprises a stretchable, self biasing material.

8. The firearm identification system of claim 1, wherein the tag comprises a stiffening member.

9. The firearm identification system of claim 1, wherein the tag comprises mating fasteners for affixing the tag in the stored, deployed and wearable positions.

10. The firearm identification system of claim 9, wherein the mating fasteners comprise hook and loop fasteners.

11. The firearm identification system of claim 1, wherein the tag has a reflective portion.

12. The firearm identification system of claim 1, wherein the tag has a portion that has enhanced visibility with night vision or thermal imaging equipment.

13. The firearm identification system of claim 1, wherein the tag has visible indicia to aid identification of the firearms bearer.

14. A method for identifying an authorized firearm bearer, comprising:
affixing a firearm identification tag to a firearm; and
selectively orienting the identification tag in a position selected from the group consisting of:
a stored position on the firearm;
a deployed position visibly extending from the firearm, and
a worn position wherein the firearm bearer wears at least a portion of the identification tag.

15. The method of claim 14 further comprising selectively wrapping the identification tag about a limb of a person as a tourniquet.

16. A method for identifying an authorized firearm bearer, comprising:
affixing a firearm identification tag to a firearm;
retaining the firearm in a holster;
selectively orienting the identification tag in a position selected from the group consisting of:
a stored position on the firearm that is visible while within the holster;
a deployed position visibly extending from the firearm, and
a worn position wherein the firearm bearer wears at least a portion of the identification tag.
